(19) United States
(12) Patent Application Publication
(10) Pub. No.: US 2019/0105272 A1
(43) Pub. Date: Apr. 11, 2019

(54) SYSTEM AND APPLIANCE FOR EXTENDING THE PRODUCTION OF NITRIC OXIDE BY SPINACH EXTRACT MICROENCAPSULATED POWDER AND ACIDIC GEL

(71) Applicant: HEBEI JINGDING BIOMEDICINE TECH. LTD., Xingtai City (CN)

(72) Inventor: CHENG SHING CHEN, Taichung City (TW)

(21) Appl. No.: 15/729,826

(22) Filed: Oct. 11, 2017

(51) Int. Cl. A61K 9/14; A61K 9/06; A61K 9/00; A61K 36/21; A61K 47/22; A61K 47/38

(52) U.S. Cl. CPC A61K 9/148; A61K 9/146; A61K 9/06; A61K 47/38; A61K 36/21; A61K 47/22; A61K 9/0002

(57) ABSTRACT

Disclosed are a method and an appliance for extending the production of nitric oxide by microencapsulated spinach extract powder and acidic gel. The appliance includes a microencapsulated spinach extract acting as a nitric oxide donor, and an acidic gel having a sufficient acidity to transform the spinach extract into nitric oxide. The method and appliance for extending the production of nitric oxide by spinach extract microencapsulated powder and acidic gel can release NO for a long time and provide a long-term effect to the affected position.

### FIELD OF INVENTION

The present invention relates to the field of medicine, in particular to a microencapsulated drug containing a spinach extract used as an active ingredient, and a system and appliance including the microencapsulated drug containing the spinach extract.

### BACKGROUND OF INVENTION

### 1. DESCRIPTION OF THE RELATED ART

In mammals, nitric oxide (NO) also known as nitrogen monoxide is an endogenous physiological regulation substance generally found in physiological processes of nervous system, immune system and cardiovascular system, and has the effects of relaxing vascular smooth muscles, and causing vasodilatation artery and increased blood flow. NO is a neurotransmitter related to the activity and functions of neuron, which covers the avoidance of learning as well as the erection of male and female sex organs (Kim et al., J. Nutrition 134 (2004) 2873S). NO also has the effect of partially regulating macrophage for the cytotoxicity of microbial and tumor cells. In addition to the normal physiological functions, NO also relates to different pathophysiological conditions such as infectious shock, hypertension, stroke, and neurodegenerative disease.

NO is applied in various forms of pharmacology, including wound healing for trauma and burns, hair growth, impotence, and application to position requiring vascular dilation (such as circulating a patient's peripheral blood flow for diabetes or other injuries, and maturing the cervix during pregnancy). Although NO has physiological activity, yet it is chemically unstable in air or in human body. Therefore, its conventional pharmacological application almost takes place through a chemical reaction of various individual stable precursor compounds. In general, organic and inorganic nitrates are used as NO donors. In some of the applications, the dosage requirement of NO is low and long-lasting. NO acting as a powerful antiseptic for bacterial with a drug resistance to antibiotics needs to extend the contact time of NO with skin in antiseptic or other applications. In the antiseptic application, NO requires a very little dosage such as several parts per million (ppm) for treatment (Refer to Ghaffari et al., Nitric Oxide Biology and Chemistry 14 (2006) 21-29)), but the effectiveness of NO depends on its contact time with skin (Ormerod et al., BMC Research Notes 4 (2011) 458-465).

In spite of the prior arts as disclosed in P.R.C. Pat. Application Nos. CN201310355902.2 and CN201310356220.3) have used microencapsulated nitrite and acidified hydrogel for the system and method of extending the production of nitric oxide, the toxicity is very large since nitrite with a specific toxicity is used as a NO donor, particularly when the dosage is large. In addition, the application of the delay system and operation method relies on one or more water molecules to start the volume, and there is a limitation in the operation.

### 2. SUMMARY OF THE INVENTION

In view of the aforementioned deficiencies of the prior art, the present invention overcomes the drawbacks by providing a system for producing nitric oxide by spinach extract microencapsulated powder and acidic gel, and the system adopts a spinach extract as the NO donor and its source comes from natural plants, and the natural ingredient can avoid potential hazards caused by using nitrite as the NO donor.

The present invention also relates to a whole-set appliance for producing nitric oxide by spinach extract microencapsulated powder and acidic gel.

The present invention further provides a method and an appliance for extending the production of nitric oxide by spinach extract microencapsulated powder and acidic gel. The system for delaying the production of nitric oxide in accordance with the present invention ensures a continuous release of NO for a long time and a simple operation for the continuous release effect.

The present invention also relates to a whole-set appliance for extending the production of nitric oxide.

The system and whole-set appliance of the present invention has the features of a simple manufacturing process, and the system has a high biosafety and is capable of maximizing the physiological activity of NO for a long term.

### BRIEF DESCRIPTION OF THE DRAWINGS

FIG. 1A is a cross-sectional view of a pad containing a mixture of microencapsulated reagents (reacted to form NO) in accordance with a preferred embodiment of the present invention;

FIG. 1B is a cross-sectional view of a pad containing an internal component for maintaining the appropriate position of particles in accordance with a preferred embodiment of the present invention;

FIG. 1C is a cross-sectional view of a pad with an absorbing layer containing a microencapsulate reagent (reacted to form NO) in accordance with a preferred embodiment of the present invention;

FIG. 2 shows a release process of NO in a solution of a microencapsulated spinach extract and an acidic gel for a release time of 5 hours continuously;

FIG. 3 shows a release process of NO in a paste of a microencapsulated spinach extract and an acidic gel for a release time more than 5 hours continuously;

FIG. 4 shows a release process of NO in a paste of a microencapsulated spinach extract and an acidic gel for a release time of 8 hours continuously; and

FIG. 5 shows a release process of NO in a paste of a microencapsulated spinach extract and an acidic gel containing a reducing agent for a release time of 8 hours continuously.

### DESCRIPTION OF THE PREFERRED EMBODIMENTS

To make it easier for our examiner to understand the objective of the invention, its structure, innovative features, and performance, we use a preferred embodiment together with the attached drawings for the detailed description of the invention.

The present invention discloses a method and an appliance for extending the production of nitric oxide by spinach extract microencapsulated powder and acidic gel, which is also a system of delaying the release of nitric oxide comprises a microencapsulated spinach extract and an acidic gel, wherein the acidic gel comes with a sufficient acidity to transform the spinach extract into nitric oxide.

The present invention adopts a spinach extract to produce a pharmaceutically acceptable NO donor. The following chemical formula (1) shows the principle of the reaction of the present invention, wherein a nitrite is reacted with an acid (HA) to form another nitrite. The nitrite is stable in water solution at a low temperature, but it will be decomposed into NO and NO₂ easily at room temperature as shown in the following chemical formula (2).

With the existence of a reducing agent (such as an ascorbic acid, a dihyroxy ascorbic acid (Asc(OH)₂)), and NO₂ is transformed into NO very easily as shown in the following chemical formula (3).

2HA+NaNO₂→2HNO₂+2NaA (1)

Where, HA is an organic acid or an inorganic acid.

2HNO₂→NO+NO₂+H₂O (2)

The nitrite is decomposed to form nitrogen dioxide.

NO+NO₂+H₂O+Asc(OH)₂→2NO+2H₂O+AscO₂ (3)

The ascorbic acid is reacted to form nitric oxide.

The present invention uses the spinach extract as the NO donor to reduce the toxicity of the nitrite, and such toxicity is a potential hazard to human body.

In an embodiment, the spinach extract keeps vitamin C in spinach, so that the system and whole-set appliance of the present invention still can keep releasing NO for a long time without adding a reducing ingredient. The vitamin C in the spinach extract has a reducing power for preventing or retarding the nitric oxide from being oxidized into nitrogen dioxide and also the power of reducing NO₂ into NO directly, so that the gas released from the composite is mainly NO.

In an embodiment, the spinach extract is used in a spinach extraction process, and an acid is provided for regulating the solution in each extraction step to a pH value of 3˜4 to ensure the extraction of vitamin C from the spinach and the activity of vitamin C.

The spinach extract of the present invention is extracted from a conventional method, and the extraction process is regulated with a pH value of 3˜4 to ensure the extraction of vitamin C and the activity of vitamin C.

In an embodiment, the spinach extract is used in a spinach extraction process, wherein the acid with the regulated pH value is oxalic acid.

In an embodiment, the spinach extract has a nitrite content of 5.017×10⁻³ mg/ml, a nitrate content of 0.236 mg/ml, and a vitamin C content of 1.21×10⁻² mg/ml.

In an embodiment, the microencapsulated carrier is a polymer matrix. The reagent and matrix are disposed into a sub-millimeter structure (at least having one scale less than 1 millimeter). Such structure may be a particle, a fiber, or a film.

In an embodiment of the present invention, a microencapsulated spinach extract is in contact with acidified hydrogel having a sufficient acidity to transform a nitrite into nitric oxide. Although an inorganic acid such as boric acid is suitable, yet a preferred acidifier is an organic acid such as citric acid. Other acidifiers may include lactic acid, glycerol acid, formic acid, ascorbic acid or any other organic acid well-known by people having ordinary skill in the art. Any biologically acceptable inorganic acid with an appropriate pKa value (such as the aforementioned boric acid) may be used. The gel includes hydroxymethyl cellulose, hydroxylethyl cellulose, gelatin, agar, natural gum, starch and pectin.

The medium for dissolving acid may be an aqueous medium or a non-aqueous medium. Preferably, an aqueous medium is used to prepare the gel easily. The acidic gel composite further includes one or more acidic conjugate bases. Although a preferred base is an acidic conjugate base, yet other organic bases or inorganic bases well known to people having ordinary skill in the art may be used. The embodiment of the present invention may be applied directly to achieve the effects of promoting the loop of skin, expediting wound healing, providing a treatment on a scalp for a period of time in order to grow hair, and releasing NO to a required position.

In an embodiment of the present invention, the gel includes a reducing agent for further assisting the biological activity of nitric oxide. The acidifier may also be a reducing agent such as an ascorbic acid (vitamin C) or an ascorbic acid derivative. The ascorbic acid derivative includes but not limited to 3-O-ethyl ascorbic acid and other 3-alkyl ascorbic acid, 6-O-octyl ascorbic acid, 6-O-dodecanoyl-ascorbic acid, 6-O-tetradecanoyl-ascorbic acid, 6-O-octadecyl -ascorbic acid and 6-O-decadecyl-ascorbic acid. A preferred reducing agent together with the vitamin C in the spinach extract have the reducing power for preventing or retarding nitric oxide from being oxidized into nitrogen dioxide, and also have the power of reducing NO₂ into NO directly, so that the gas released from the composite is mainly NO. A preferred reducing agent includes an ascorbic acid, an ascorbic acid derivative, an ascorbic acid salt, tocopherol, isoascorbic acid, or a-tocopherol.

An embodiment of the present invention also discloses a set of acidified gel and microencapsulated spinach extract, wherein both acidified gel and microencapsulated spinach extract are packaged into different moisture-proof packages respectively. In an application, the packages are opened, and their contents are mixed. In another embodiment, the microencapsulated spinach extract and acidifier are packaged into moisture-proof package(s) together or separately, and the package(s) are opened and a specific quantity of water or aqueous gel with a neutral pH is mixed with the microencapsulated spinach extract and acidifier.

Microencapsulation is a manufacturing method, wherein a reagent of a dissolved solution or a polymer solution is atomized and dried to produce a powder with tiny scattered individual particles (containing the reagent scattered in the polymer matrix). Other microencapsulation methods such as pan coating, air suspension coating, centrifugal extrusion, fiber spinning, fiber extrusion, nozzle vibration, ion gel, condensation phase separation, interface cross-linking, in situ polymerization, and matrix polymerization may be used.

In a method for manufacturing microencapsulated spinach extract in accordance with a preferred embodiment of the present invention, a stable agent is added into the spinach extract, and the stable agent is capable of maintaining the activity of vitamin C for a long time during the microencapsulation manufacturing process, wherein the stable agent is L-cysteine hydrochloride and sodium metabisulfite.

To cope with the applicability for medical indications, the present invention discloses a packaged polymer which is a biocompatible polymer. An appropriate polymer includes ethyl cellulose, natural polymers such as corn protein (which is a certain protein found in a gramineous plant such as a corn and a grain and stored in alcoholic seeds), deacetylated chitosan, hyaluronic acid, alginic acid, biodegradable polyester, polyanhydride, polyethylene (ortho ester), polyphosphazene or polysaccharide (See Park et al., Molecules 10(2005)146-161).

The microencapsulated composite of the reagent used for transmitting a medical reagent is well-known in the art and has been disclosed by Shalaby and Jamiolkowski, U.S. Pat. No. 4,130,639; and Buchholz and Meduski, U.S. Pat. No. 6,491,948, etc. However, the microencapsulated reagents of these composites are treatment agents, and the treatment agents are not produced by the reaction of the microencapsulated reagent. Related medical journals have disclosed the nitric oxide release polymer in a nitric oxide compound/donor. For example, Arnold, U.S. Pat. No. 7,829,553 (diol dinitenly carbonyl groups are attached onto hydrophobic polymers); and Knapp, U.S. Pat. No. 7,135,189 (s-nitrosothiols precursor and nitric oxide donor).

In an embodiment of the present invention, the microencapsulated reagent is applied directly for transmitting NO to wound, wound dressing, surgical dressing, bed guards, and socks of decubitus patients (or patients to be), clothing and orthopedic gypsum of diabetes and other loop dysfunction patients, and vasodilators for the treatment of sexual dysfunction. The present invention also can meet the requirements for a small quantity and a long-lasting NO dosage related to routine implantation or insertion for medial appliances (such as vascular stents, catheters, pacemakers, defibrillators, cardiac assist devices, artificial valves, electrodes, and orthopedic screws and pins).

The present invention may be a wound dressing pack or a bandage pack, and a portion of the wound dressing includes particles of a microencapsulated reagent. Such dressing is also combined with a material with a water retention function for maintaining the required appropriate amount of water when the particles are situated in a humid environment. The dressing is wetted to initiate the reaction of the reagent, and the dressing starts releasing NO. The dressing is designed to release NO near the wound.

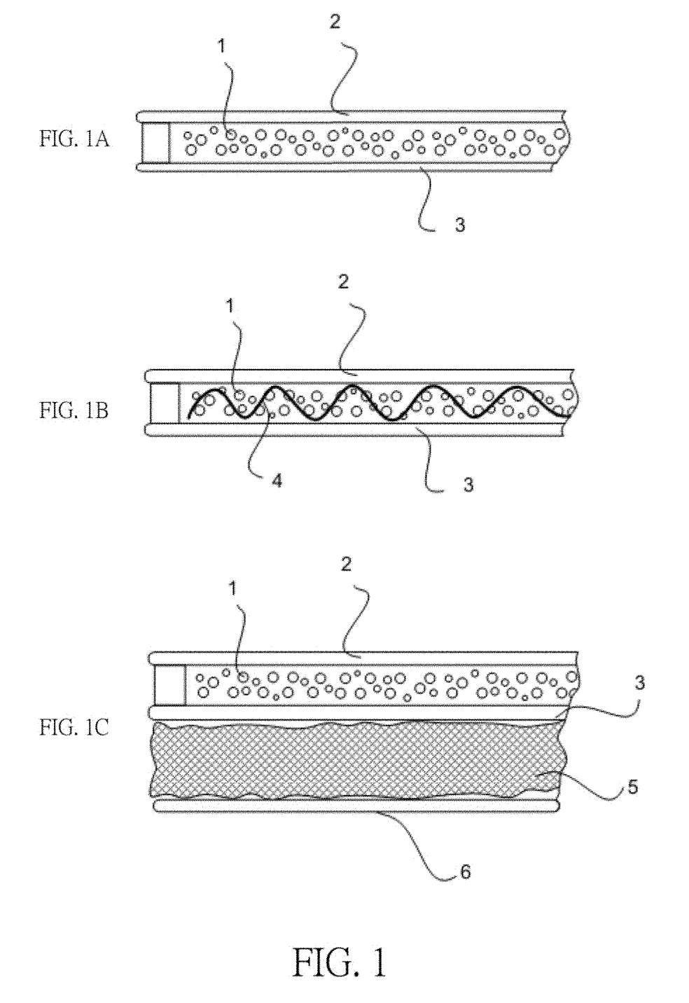

An embodiment of the present invention discloses a multifunctional technology for delaying the release of NO through a layered pad. With reference to FIG. 1A for a cross-sectional view, the particles 1 are included between a layer 2 and a layer 3, wherein at least one side of the layers 2, 3 faces the body layer to transmit gaseous NO, while at least one side faces an outer layer, and such side facing the outside layer has the impermeable and water-retention functions (and allows an applied liquid to be transmitted into the particles and/or maintains the particles situated in a humid environment). In an application of supplying NO on a desired side of the pad, one of the layers 2 and 3 does not permeate NO. The sub-millimeter particle includes the microencapsulated spinach extract. In the environment of an aqueous solution, the particles are transformed to form a reagent and combined to produce NO. When water is introduced to the pad, the reagent starts the release to produce NO.

In an embodiment as shown in FIG. 1B, the outer layers 2, 3 are separated by an isolation layer 4, and the isolation layer 4 is provided for maintaining the distance between the outer layers 2, 3 and the appropriate position of the particles in the layer. The particles containing the reagent may be fixed onto the isolation layer 4 or on an inner surface of any one of the outer layers 2, 3 by embedment or any other fixing method.

The pad as shown in FIG. 1 may be manufactured to any specific size and shape. In FIGS. 1A˜C, the vertical size is not drawn in a proportional scale. A water absorbent material 5 may be much thicker than the pad containing the reagent.

Therefore, the aforementioned pad is multifunctional, wherein the pad may be placed on a wound adhered to a layer of a medical tape for a simple application; the pad may be pre-made into a bandage or dressing. Preferably, the bandage or dressing is equipped with a small pack, and the small pack contains a microencapsulated reagent which can be reacted to form NO. In addition, the reagent may be attached to layers of different materials and then assembled to produce the bandage or dressing.

The pad of another structure as shown in FIG. 1 may be a long-lasting antibacterial wiping cloth, and the size of such pad may be change to fit its insertion into the clothing such as socks or tight pants for loop dysfunction patients. The edge of the material and the pad structure containing the particles are processed appropriately, the pad itself may be the woven socks, gloves, and clothes for loop dysfunction patients. The aforementioned clothing may be initiated by the natural moisture coming from the patient's skin or initiated by added water.

With reference to FIG. 1C for a cushion in accordance with another embodiment of the present invention, the cushion comprises a pad containing particles, an absorbing layer or a permeating layer 5, and an impermeable layer 6 disposed under the aforementioned layers. The absorbing cushion is applicable for decubitus patients or patients to be. These patients produce an appropriate amount of water through urinary incontinence and sweat. The moisture initiates the pad that produces NO, and the extra moisture is absorbed by the absorbing layer under the pad. Such arrangement allows decubitus to be bathed in NO and the nitric oxide stimulates the decubitus healing and prevents the ulcer area from expanding further.

In different applications, a small dosage of NO may be applied to a male rat's penis to erect the penis quickly, and it shows that the effect is very effective (Han et al., Journal of Sexual Medicine 7 (2010) 224). The present invention has discloses an application of the similar effect to human beings by applying the NO. At present, systemic drugs for sexual dysfunction have side effects, and take some time to take effect. As to the controllability and unfound systemic side effects, drugs with such quick and topical treatment effect are required. The reagent capable of producing NO may be put on a dried coating of a dressing of an erectile tissue. In an embodiment, the reagent may be applied to an internal dressing of a male or female condom, and the dressing of the erectile tissue is wetted to initiate the reagent in order to delay the release of NO.

In another embodiment of the present invention, an inner surface of the condom is coated with a coating, and the coating includes a microencapsulated reagent. In an aqueous solution, the microencapsulated reagents are reacted to produce NO. In this embodiment, the particle has a size from 0.01 to 100 microns, preferably 1˜10 microns. A smaller particle is favorable for attaching the coating onto the inner surface of the condom, and the NO release time is in the scale of minutes, but not hours. In this embodiment, before a user wears the condom, a water-containing compound such as K-Y jelly (manufactured by McNEIL-PPC, Inc., Ft. Washington, Pa.) is applied onto the erectile tissue. When the particles are contacted with the water-containing compound, NO is released. The released NO is limited in the condom until it is absorbed by the erectile tissue through the skin and used for stimulating and extending an erection.

In another embodiment of the present invention, a sexual arousal gel reagent package comprises a hydrogel compound package containing a K-Y jelly like material and a moisture-proof package containing a microencapsulated reagent, wherein the reagent in an aqueous solution is reacted to produce NO. Before use, the packages are opened and mixed to the aqueous gel, and the mixture is applied to a male/female user's external genitialia to stimulate the blood circulation and promote the erection of the penis and clitoris. Such package may be used for the treatment of sexual dysfunction and the enhancement of male or female's sexual life satisfaction.

Although there is no human clinical research, the research on rats contacted by Seitz, et al (U.S. Pat. No. 6,103,275) shows that the gel composite can stimulate hair growth. As we all know, local vascular dilation drugs such as minoxidil can alleviate human hair loss and stimulate the growth of hair, so that a long- lasting and low dosage of NO (NO is an effective vasodilator), and a local application is highly probable to have good treatment effect for hair loss. Therefore, this invention discloses another application on an apparatus and a composite for delaying the release of NO to alleviate hair loss and stimulate the growth of hair. In an embodiment, the material as shown in FIG. 1 is used to manufacture a head-shaped hat for treating hair loss, and the hat is manufactured to fit the bald area of the patient's head and water is provided to wet the hat and initiate its function.

Exemplary embodiments are provided for the purpose of illustrating the advantages and features of the present invention, but not intended for limiting the scope of the invention. While the invention has been described by means of specific embodiments, numerous modifications and variations could be made thereto by those skilled in the art without departing from the scope and spirit of the invention set forth in the claims.

The chemical reagents used for the analysis in the embodiments of the present invention are bought from Sinopharm Group Co., Ltd.

To make it easier for our examiner to understand the technical characteristics of the present invention, the following embodiments are described, wherein the experiment method of the present invention are normal and the biomaterial are commercially available, unless specified otherwise.

### Embodiment 1: Preparation of Spinach Extract

1.1 Extraction of Spinach

The edible part of the spinach is washed by tap water and then deionized water, dried, and finally cut into small pieces. 10 g of the cut spinach are put into a big beaker, and 50 mL of the deionized water is added, and an oxalic acid is used to regulate the pH value of the extract to 3˜4, and then grounded. The extract is put and incubated in a water bath at 70□ for 30 minutes, and the extract is filtered into a 100 mL flask, and the deionized water is used for fixing the volume to 100 mL, and the oxalic acid is used to regulate the pH value of the extract to 3˜4, and then concentrated to approximately 10 mL, and the concentrated extract is transferred into a 250 mL beaker, and 5 mL of saturated borax solution and 100 mL of hot water (at 70˜80□) are added, and the oxalic acid is used to regulate the pH value of the extract pH to 3˜4, and the extract is put into a boiled water bath and heated for 15 minutes while shaking continuously, and then removed and cooled to room temperature, and 10 mL of potassium ferrocyanide solution, 10 mL of zinc acetate solution, and 2 g of activated carbon powder are added, and the solution is mixed thoroughly while adding, and then the mixed solution is transferred into a 250 mL flask, and the oxalic acid is used to regulate the pH value of the extract to 3˜4, and water is used to fix the volume, and the extract is filtered to obtain a colorless and clear solution.

In the extraction process, L-cysteine hydrochloride and sodium metabisulfite are added to ensure the activity of vitamin C, wherein the L-cysteine hydrochloride content is 0.1%, and the sodium metabisulfite content is 0.2%.

1.2 Measurement of Nitrate

The nitrite content and the nitrate content of the spinach extract are measured by spectrophotometry, and the result shows that spinach extract has a nitrite content of 5.017×10^{'3} mg/ml and a nitrate content of 0.236 mg/ml.

The vitamin C content of the spinach extract is measured by spectrophotometry, and the result shows that the spinach extract has a vitamin C content of 1.21×10⁻² mg/ml.

### Embodiment 2: Preparation of Microencapsulated Spinach Extract

The spinach extract as prepared in Embodiment 1 is concentrated, and a solution composed of the spinach extract, L-cysteine hydrochloride and sodium metabisulfite (vitamin C stable agent), and a solution consisting of corn gliadin and a volatile solvent are used to prepare spinach extract particle with the corn gliadin as a matrix and containing a nitrite of 10% (percentage by weight). The corn gliadin is a proline-rich acidic protein obtained from a corn and acts as a coating and a packaged matrix used for processed food and medicines. The corn gliadin is listed by U.S. Food and Drug Administration USFDA as Generally Recognized as Safe (GRAS). The solution contains 10% of corn gliadin (Flo Chemicals, 29 Puffer St., Ashburnham, Mass. 01430 (Lot F4000011106)) is scattered in a mixture of ethanol and water (mixed in a ratio of 90:10). The solution further contains a stable agent L-cysteine hydrochloride and sodium metabisulfite (vitamin C stable agent), an L-cysteine hydrochloride content of 0.1%, and a sodium metabisulfite content of 0.2%, and the solution is scattered into a dryer with a rotary disc atomizer, and the particles of this sort have a particle diameter from 10 to 100 microns, and these particles include the corn gliadin matrix scatted in the spinach extract. The corn gliadin is insoluble in water, so that when the particles are exposed from water, the water is slowly diffused into the corn gliadin matrix to dissolve sodium nitrite and vitamin C in the spinach extract, and the solution containing sodium nitrite is slowly diffused from the particles to provide a continuous release of sodium nitrite for a longer time.

### Embodiment 3: Release of NO from Microencapsulated Spinach Extract and Acidic Gel in Solution

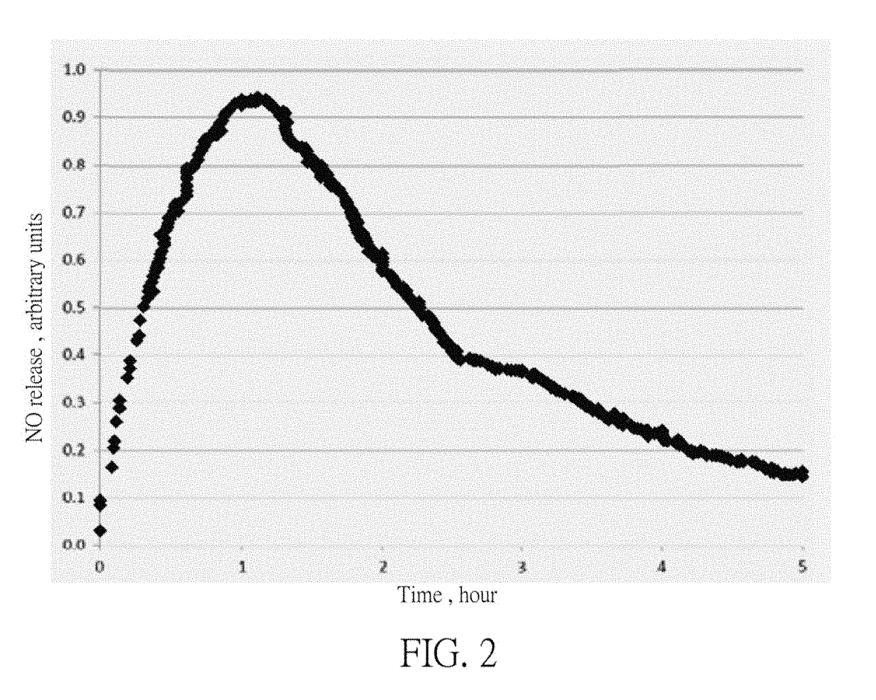

An aqueous solution (100 ml) containing 5.6 g of citric acid and 0.3 g of PE9010 (which is a preservative manufactured by Schü and Mayr, 30 Two Bridges Road Suite 225, Fairfield, N.J. 07004, USA) is prepared. The solution (40 ml) is put into a beaker, and an inNO-T nitric oxide measurement system (by Innovative Instruments, Inc., Tampa, Fla. 33637) equipped with an amiNO-700 probe is used to measure the concentration of NO in the solution. 10 mg of the particles of the microencapsulated spinach extract containing the corn gliadin as prepared in Embodiment 2 is added into the solution at a specific time (0 hour), and the NO content in the solution is recorded, and the production of NO starts after the particles are added. After approximately 20 minutes, NO is produced from the liquid, and the quantity of NO is increased gradually until it reaches the peak value after approximately an hour, and then the quantity of NO is decreased gradually. The whole NO release time lasts for 5 hours, and sodium nitrite and the acid in the solution for producing NO by the microencapsulated particles are reacted according to Chemical formulas (1) to (3), and the release process of NO is shown in FIG. 2.

### Embodiment 4: Release of NO from Microencapsulated Spinach Extract in Solution

The particles (10 mg) as prepared in Embodiment 2 are put into a container. The mixture is added into a beaker containing 40 ml of deionized water and blended uniformly.

An inNO-T nitric oxide measurement system (by Innovative Instruments, Inc., Tampa, Fla. 33637) equipped with an amiNO-700 probe is used for measuring the concentration of NO in the solution, and the tip of the amiNO-700 probe is added into the solution at a specific time (0 hour), and the NO content in the solution is recorded, and then the NO signal is recorded. A tiny amount of NO can be detected within the first three hours of monitoring.

### Embodiment 5: Release of NO from Microencapsulated Spinach Extract and Acidic Gel in Paste

This embodiment intends to simulate a NO release process when equal amounts of microencapsulated spinach extract and acidic gel are applied directly to a patient's body surface.

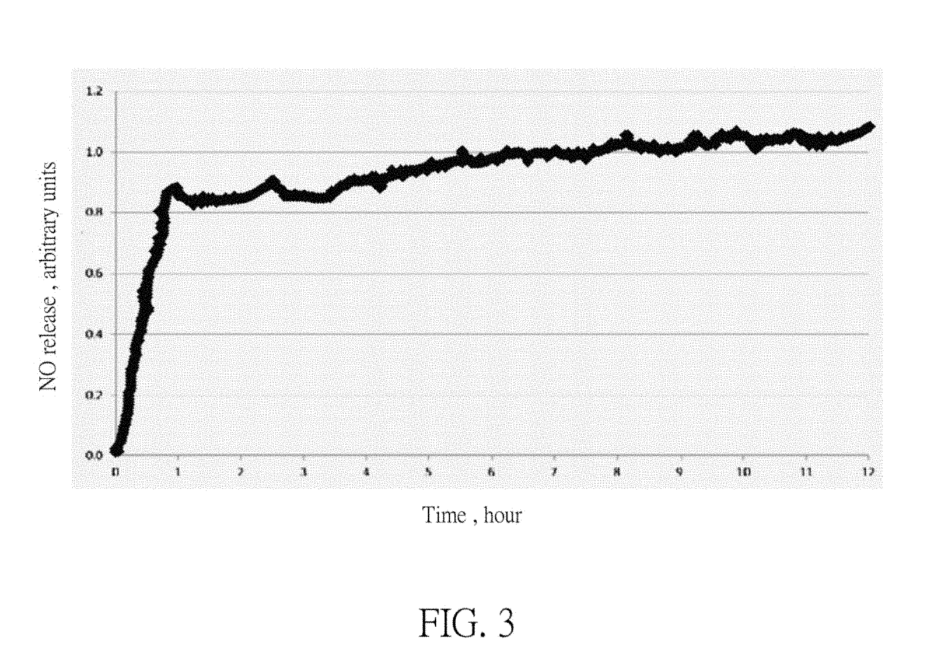

The particles (10 mg) as prepared in Embodiment 2 are put onto a weighing paper having a small fold. The tip of the amiNO-700 probe is inserted and covered completely with a powder mixture. An equal quantity of the acidic gel and an equal quantity of the microencapsulated spinach extract as prepared in Embodiment 3 are mixed uniformly, and the NO signal is recorded, a trace of deionized water may be added during the recording process according to the drying condition of the paste system timely. In the whole NO release, the recording starts at the beginning, and the NO release reaches a relatively higher level after approximately 1 hour after the start, and the whole release process takes more than 10 hours. During the process, the level of NO is always situated at a stable and steady status. Refer to FIG. 3 for the NO release process.

### Embodiment 6: Release of NO from Microencapsulated Sodium Nitrite and Acidic Gel in Paste

According to the microencapsulation method of Embodiment 2, a solution composed of sodium nitrite, ethyl cellulose, and a volatile solvent is atomized and dried to prepare particles of sodium nitrite using ethyl cellulose as the matrix (wherein sodium nitrite has a percentage by weight of 10%).

The microencapsulated sodium nitrite particles (10 mg) are put onto a weighing paper with a small fold. The tip of the amino-700 probe is inserted and covered completely with a powder mixture.

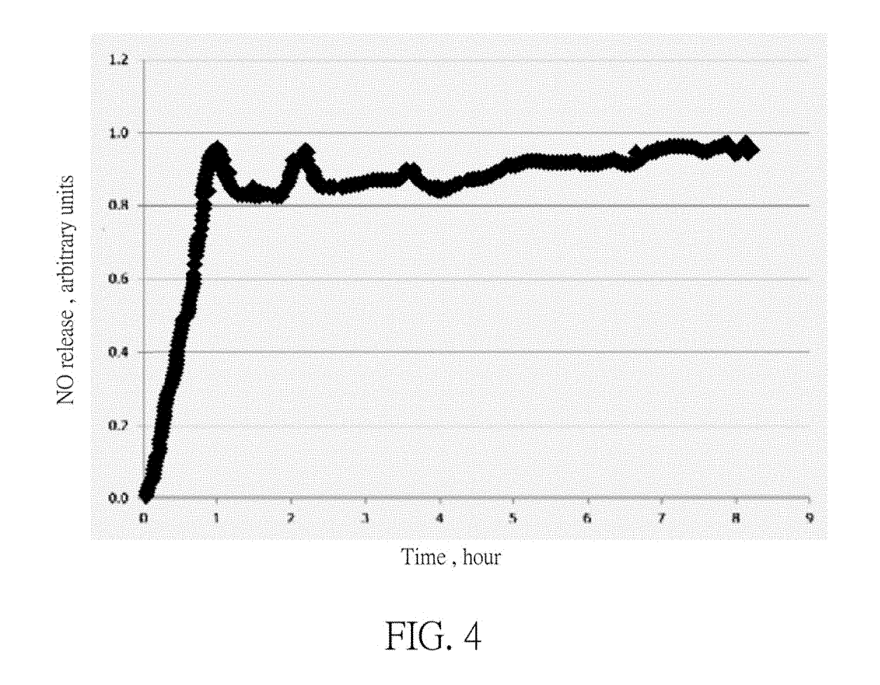

An equal quantity of the acidic gel and an equal quantity of the microencapsulated spinach extract as prepared in Embodiment 3 are mixed uniformly, and the NO signal is recorded, a trace of deionized water may be added during the recording process according to the drying condition of the paste system timely. In the whole NO release, the recording starts at the beginning, and the NO release reaches a relatively higher level after approximately 1 hour after the start, and the whole release process takes 8 hours. During the process, the level of NO is always situated at a stable and steady status. Refer to FIG. 4 for the whole NO release process.

### Embodiment 7: Release of NO from Microencapsulated Spinach Extract and Acidic Gel in Paste, and the Acidic Gel further Includes a Reducing Agent

This embodiment intends to simulate the NO release process by applying an equal quantity of microencapsulated spinach extract and an equal quantity of acidic gel directly onto a patient's body surface, and the prepared acidic gel includes an additional reducing agent (vitamin C).

An aqueous solution (100 ml) is prepared, and the aqueous solution includes 5.6 g of citric acid, 2.2 g of ascorbic acid, and 0.3 g of PE9010 (which is a preservative manufactured by Schü and Mayr, 30 Two Bridges Road Suite 225, Fairfield, N.J. 07004, USA).

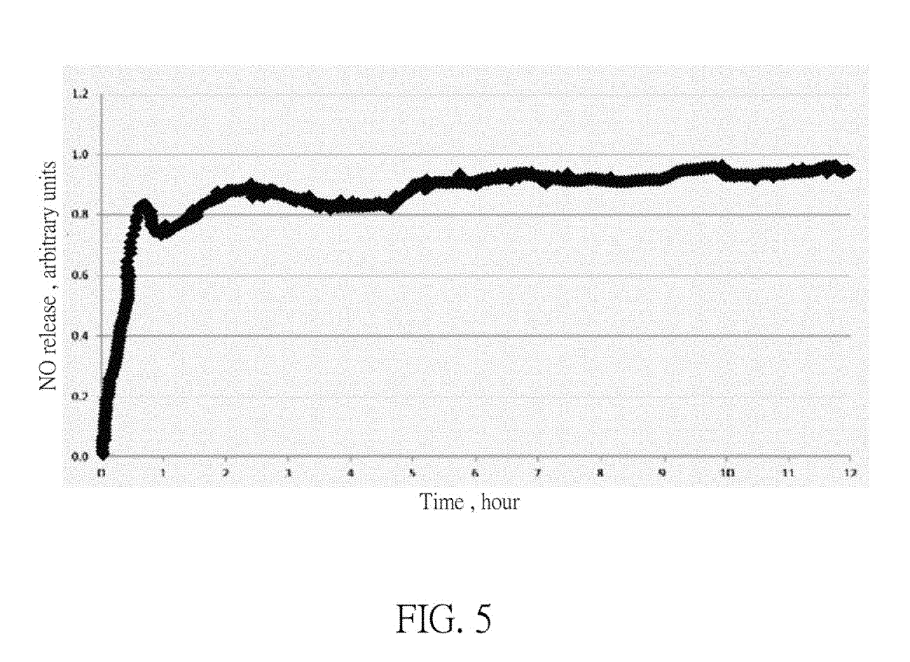

The particles (10 mg) are put onto a weighing paper with a small fold. The tip of the amiNO-700 probe is inserted and covered completely by a powder mixture. An equal quantity of the aforementioned acidic gel containing the additional reducing agent is mixed with the microencapsulated spinach extract, and then the NO signal is recorded. A trace of deionized water may be added during the recording process according to the drying condition of the paste system timely. In the whole NO release, the recording starts at the beginning, and the NO release reaches a relatively higher level after approximately 40 minutes after the start, and the whole release process takes more than 10 hours. During the process, the level of NO is always situated at a stable and steady status. Refer to FIG. 5 for the NO release process.

The experiment result shows that the microencapsulated spinach extract of the present invention can provide a longer NO release time than the microencapsulated nitrite (wherein the microencapsulated nitrite can maintain 8 hours of NO release). Even if no reducing agent is added into the system, the system will be able to maintain the release of NO with a biologically effective concentration for more than 10 hours in the process, and the speed of the NO release is constant.

The aforementioned factors are analyzed, the composite structure of the nitrite and vitamin C existed in a specific form in the spinach extract or other substance can maintain a longer NO release time during the NO release process.

What is claimed is:

1. A system for delaying the release of nitric oxide, comprising a microencapsulated spinach extract, and an acidic gel with a sufficient acidity to transform the spinach extract into nitric oxide.

2. The system for delaying the release of nitric oxide according to claim 1, wherein the spinach extract contains vitamin C coming from spinach.

3. The system for delaying the release of nitric oxide according to claim 1, wherein the acidity is provided by one or more organic acids selected from the group consisting of citric acid, lactic acid, glycerol acid, formic acid, and ascorbic acid.

4. The system for delaying the release of nitric oxide according to claim 1, wherein the acidity is provided by boric acid.

5. The system for delaying the release of nitric oxide according to claim 1, wherein the acidic gel includes one or more polymers selected from the group consisting of hydroxymethyl cellulose, hydroxyetthyl cellulose, gelatin, agar, natural gum, starch and pectin.

6. The system for delaying the release of nitric oxide according to claim 1, wherein the acidic gel includes a specific quantity of polymer, and the polymer solution has a viscosity equal to the viscosity of 0.7-3.0% (w/v) hydroxyetthyl cellulose aqueous solution.

7. The system for delaying the release of nitric oxide according to claim 1, wherein the acidic gel further includes a reducing agent, and the reducing agent includes an ascorbic acid (vitamin C) or an ascorbic acid derivative; and the ascorbic acid derivative includes 3-O-ethyl ascorbic acid and 3-alkyl ascorbic acid, 6-O-octyl ascorbic acid, 6-O-dodecanoyl-ascorbic acid, 6-O-tetradecanoyl-ascorbic acid, 6-O-octadecyl-ascorbic acid, and 6-O-decadecyl-ascorbic acid.

8. The system for delaying the release of nitric oxide according to claim 1, wherein the microencapsulated spinach extract has a capsule made of ethyl cellulose, corn gliadin, deacetylated chitosan, hyaluronic acid, alginic acid, biodegradable polyester, polyanhydride, polyethylene (ortho ester), polyphosphazene or polysaccharide.

9. A whole-set appliance for delaying the release of therapeutic nitric oxide for a patient, comprising a microencapsulated spinach extract, and an acidic gel with a sufficient acidity to transform the spinach extract into nitric oxide, and the microencapsulated spinach extract and the acidic gel being placed separately.

10. The whole-set appliance for delaying the release of therapeutic nitric oxide for a patient according to claim 9, wherein the microencapsulated spinach extract is placed on a wound dressing or a bandage, and the acidic gel is placed in another container.

11. The whole-set appliance for delaying the release of therapeutic nitric oxide for a patient according to claim 9, wherein the microencapsulated spinach extract is coated onto an inner surface of a condom, and the acidic gel is placed in another container.
